(19) United States
(12) Patent Application Publication
(10) Pub. No.: US 2026/0066064 A1
(43) Pub. Date: Mar. 5, 2026

(54) SYSTEMS, DEVICES, AND METHODS FOR AUTOMATIC CONFIGURATION OF MEDICAL DATA SOURCES, MEDICAL DATA SINKS, AND NETWORK ADAPTERS

(71) Applicant: Stryker Corporation, Portage, MI (US)

(72) Inventors: Eric Alexander HEREFORD, North Richland Hills, TX (US); Neel MISTRY, Haslet, TX (US); Kyle Jason SIMONS, McKinney, TX (US)

(73) Assignee: Stryker Corporation, Portage, MI (US)

(21) Appl. No.: 19/316,663

(22) Filed: Sep. 2, 2025

Related U.S. Application Data
(60) Provisional application No. 63/690,225, filed on Sep. 3, 2024.

(51) Int. Cl. G16H 10/00; G16H 40/67

(52) U.S. Cl. CPC G16H 10/00; G16H 40/67

(57) ABSTRACT

A method for controlling use of medical data includes, at a medical room control system, querying a network adapter via a communication network to obtain information stored at the network adapter, the network adapter configured to encode medical data generated by a medical data source for transmission via the communication network and/or decode medical data received via the communication network for use by a medical data sink; determining at least one attribute associated with the network adapter, the medical data source, or the medical data sink based on the information stored at the network adapter; and controlling use of the medical data by the medical data sink based on the at least one attribute.

### CROSS-REFERENCE TO RELATED APPLICATIONS

This application claims the benefit of U.S. Provisional Application No. 63/690,225, filed Sep. 3, 2024, the entire contents of which is incorporated herein by reference.

### FIELD

The present disclosure relates generally to medical data communication and processing and, more specifically, to automatically handling medical data based on attributes of medical data sources and/or medical data sinks.

### BACKGROUND

In a medical environment, such as an operating room, multiple medical video and multimedia sources are routed to multiple video sinks, such as multiple displays. For example, video sources, such as one or more of an endoscopic camera, a camera within a surgical light (also referred to as an in-light camera), a pan-tilt-zoom camera, and a video conferencing camera may be routed to multiple different displays, such as one or more wall displays, touch screen displays, or control screens. In such environments, one or more network devices are needed to enable video signals from a given video source to be routed to one or more video sinks. These networked devices may include one or more hubs, switches, and routers. If video sources and sinks are not properly configured, then displays may not be able to properly display any video, or the wrong display may be used to display a given video feed. Conventionally, setting up proper connections within the video communications network requires a technician to determine how the video sources and sinks should be routed and how they are physically connected, and how to manually assign correspondences between the video sources and video sinks.

Manually creating connections between video sources and video sinks is not only a complex process, but also a time-consuming process occurring during installation of a video source or video sink. For example, in real-world environments, network components are tightly fitted inside racks and connected to switches through bundles of fiber cables. A technician may struggle to determine how a given video source or sink is connected to the network, which the technician may need to know to manually update a routing table. Such a process may be difficult and error prone.

Additionally, video sinks may need configuring based on which video source is providing its video feed. For example, the color space or resolution of a display may need to be adjusted based on the video feed provided by a particular video source. Configuring data sinks may require a technician knowledgeable about the capabilities of the video sources and video sinks, which may limit how quickly a given video sink can be installed. Even with an expert technician available for installation, the configuration process may be lengthy and may be error prone.

### SUMMARY

Described herein are systems, devices, and methods for automatically handling medical data usage in a networked medical room environment. Information associated with medical data sources (e.g., cameras) and/or medical data sinks (e.g., displays) may be stored in network adapters associated with the medical data sources and/or sinks, the network adapters being used to transmit and receive the medical data within the networked medical room environment. The information can be used to automatically determine how medical data should be handled. The information stored at a network adapter may include an identifier associated with a connected medical data source or medical data sink. The identifier can be used to determine one or more attributes associated with the medical data source or medical data sink connected to the network adapter, such as via a query to a database storing attributes for various medical data sources and/or medical data sinks. Alternatively, the attributes can form at least some of the information stored at the network adapter.

The information stored at a network adapter, such as an identifier for a connected medical data source or medical data sink, can be used to automatically configure routing for the medical data source or medical data sink. For example, a database storing attributes may include pre-determined routing for a given medical data source, and a database query (using an identifier of the medical data source) can be used to obtain the pre-determined routing for the medical data source. Additionally, or alternatively, a medical data source or medical data sink may be automatically configured based on attributes obtained from a local or remote database using information stored at a network adapter.

According to an aspect, a method for controlling use of medical data includes, at a medical room control system, querying a network adapter via a communication network to obtain information stored at the network adapter, the network adapter configured to encode medical data generated by a medical data source for transmission via the communication network and/or decode medical data received via the communication network for use by a medical data sink; determining at least one attribute associated with the network adapter, the medical data source, or the medical data sink based on the information stored at the network adapter; and controlling use of the medical data by the medical data sink based on the at least one attribute.

Controlling the use of the medical data by the medical data sink may include configuring the medical data source and/or the medical data sink based on the at least one attribute. The medical data source and/or the medical data sink may be configured based on a user profile. Configuring the medical data source and/or the medical data sink may include configuring a resolution and/or a color space of the medical data sink.

Determining the at least one attribute may include extracting an identifier for the network adapter, the medical data source, or the medical data sink from the information stored at the network adapter. The identifier may include a Global Trade Item Number (GTIN). Determining the at least one attribute may include using the identifier to query a database to obtain the at least one attribute. The database may be hosted by a remote computing system. The database may be stored locally at the medical room control system. The at least one attribute may include an association of the medical data source with the medical data sink, and wherein controlling the use of the medical data by the medical data sink based on the at least one attribute may include updating a routing table based on the association of the medical data source with the medical data sink so that the medical data from the medical data source is routed to the medical data sink. The network adapter may be associated with the medical data source, and the at least one attribute may include an association of the network adapter associated with the medical data source with a network adapter associated with the medical data sink, and wherein controlling use of the medical data by the medical data sink based on the at least one attribute may include updating a routing table based on the association of the network adapter associated with the medical data source with the network adapter associated with the medical data sink so that the medical data from the medical data source is routed to the medical data sink.

Determining the at least one attribute may include extracting the at least one attribute from the information stored at the network adapter. The method may include tracking a number of uses and/or a device uptime associated with the network adapter. The data stored at the network adapter may include the number of uses and/or the device uptime associated with the network adapter. The method may include generating a notification indicating maintenance associated with the network adapter based on the number of uses and/or the device uptime.

The method may include monitoring data communication signal power associated with the network adapter. The method may include generating a notification indicating maintenance associated with the network adapter based on the monitoring of the data communication signal power. The medical data source or the medical data sink is connected to the network adapter via a cable. The medical data source or the medical data sink may include the network adapter.

According to an aspect, a system for controlling display of medical video includes one or more processors and memory storing one or more programs for execution by the one or more processors, the one or more programs including instructions that when executed by the one or more processors cause the system to query a network adapter via a communication network to obtain information stored at the network adapter, the network adapter configured to encode medical data generated by a medical data source for transmission via the communication network and/or decode medical data received via the communication network for use by a medical data sink; determine at least one attribute associated with the network adapter, the medical data source, or the medical data sink based on the information stored at the network adapter; and control use of the medical data by the medical data sink based on the at least one attribute. The system may include the network adapter, the medical data source, and the medical data sink.

Controlling the use of the medical data by the medical data sink may include configuring the medical data source and/or the medical data sink based on the at least one attribute. The medical data source and/or the medical data sink may be configured based on a user profile. Configuring the medical data source and/or the medical data sink may include configuring a resolution and/or a color space of the medical data sink.

Determining the at least one attribute comprises extracting an identifier for the network adapter, the medical data source, or the medical data sink from the information stored at the network adapter. The identifier may include a Global Trade Item Number (GTIN). Determining the at least one attribute may include using the identifier to query a database to obtain the at least one attribute. The database may be hosted by a remote computing system. The database may be stored locally at the medical room control system.

The at least one attribute may include an association of the medical data source with the medical data sink, and controlling the use of the medical data by the medical data sink based on the at least one attribute may include updating a routing table based on the association of the medical data source with the medical data sink so that the medical data from the medical data source is routed to the medical data sink. The network adapter may be associated with the medical data source, and the at least one attribute may include an association of the network adapter associated with the medical data source with a network adapter associated with the medical data sink, and controlling use of the medical data by the medical data sink based on the at least one attribute may include updating a routing table based on the association of the network adapter associated with the medical data source with the network adapter associated with the medical data sink so that the medical data from the medical data source is routed to the medical data sink.

Determining the at least one attribute may include extracting the at least one attribute from the information stored at the network adapter. The one or more programs may include instructions for tracking a number of uses and/or a device uptime associated with the network adapter. The data stored at the network adapter may include the number of uses and/or the device uptime associated with the network adapter. The one or more programs may include instructions for generating a notification indicating maintenance associated with the network adapter based on the number of uses and/or the device uptime.

The one or more programs may include instructions for monitoring data communication signal power associated with the network adapter. The one or more programs may include instructions for generating a notification indicating maintenance associated with the network adapter based on the monitoring of the data communication signal power.

The medical data source or the medical data sink may be connected to the network adapter via a cable. The medical data source or the medical data sink may include the network adapter.

According to an aspect, a non-transitory computer readable medium stores instructions for execution by one or more processors of a computing system to cause the computing system to perform any of the methods described above.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1 illustrates an exemplary system operating in a medical room environment;

FIG. 2 illustrates an exemplary method for controlling use of medical data by one or more medical data sinks based on information stored at one or more network adapters;

FIG. 3 is a functional block diagram of examples of a medical data source, a medical data sink, a network adapter, and a medical room control system;

FIG. 4 illustrates an exemplary method for adding a medical data source to a system;

FIG. 5 illustrates an exemplary method for monitoring one or more aspects of the operation of a medical data source, a medical data sink, and/or a network adapter; and

FIG. 6 illustrates an exemplary computing system.

### DETAILED DESCRIPTION

Reference will now be made in detail to implementations and embodiments of various aspects and variations of the invention, examples of which are illustrated in the accompanying drawings. Various devices, systems, and methods are described herein. Although at least two variations of the devices, systems, and methods are described, other variations may include aspects of the devices, systems, and methods described herein combined in any suitable manner having combinations of all or some of the aspects described. Example embodiments will now be described more fully hereinafter with reference to the accompanying drawings; however, they may be embodied in different forms and should not be construed as limited to the examples set forth herein. Rather, these embodiments are provided so that this disclosure will be thorough and complete, and will fully convey exemplary implementations to those skilled in the art.

Described herein are examples of systems, devices, and methods for automatically handling medical data usage in a networked medical room environment. A medical room may include multiple medical data sources, multiple medical data sinks, and a network connecting the sources and sinks to enable medical data from the medical data sources to be communicated to one or more of the medical data sinks. A network adapter may be associated with each medical data source to convert medical data from the associated medical data source to a data format (e.g., packets) that can be transmitted over the network. A network adapter may be associated with each medical data sink to convert medical data in the data format transmitted over the network into a format usable by the medical data sink. The network adapters may store information that may be used to automatically control how medical data is used. For example, the information may be used to automatically control how medical data is routed within the network, how medical data is processed by a medical data sink (e.g., how it is displayed), and/or how medical data is generated by a medical data source.

A medical room control system may control automatic handling of medical data usage in a networked medical room environment. The medical room control system can query a network adapter to obtain information associated with a medical data source or sink connected to the network adapter. The information stored at the network adapter can be used by the medical room control system to determine one or more attributes associated with the medical data source or sink. The medical room control system may extract the attribute(s) from the information it receives from the network adapter (i.e., the attribute(s) are stored at the network adapter). Alternatively, the medical room control system may extract an identifier associated with the medical data source or sink and may use the identifier to obtain the attribute(s). The medical room control system may store the attributes of medical data sources and/or medical data sinks or the medical room control system may query a remotely hosted database of attributes using the identifier.

An attribute associated with a given medical data source or sink may include how medical data source or sink should be connected within the medical room environment. For example, the attribute may be an association of the medical data source with a particular medical data sink. The medical room control system can use such an attribute to update a routing table so that medical data from the medical data source is routing to the particular medical data sink. Thus, a medical data source or sink can be automatically connected within a medical room environment and its medical data routed appropriately without requiring costly and time-consuming manual routing. This may be particularly advantageous for mobile medical data sources and/or mobile medical data sinks, which can be more easily and efficiently moved between medical rooms.

An attribute associated with a given medical data source may be used by the medical room control system to automatically configure a medical data sink. For example, a resolution or color space attribute of the medical data source can be used to adjust a resolution or color space configuration of the medical data sink (e.g., display) to which medical data from the medical data source is routed. Additionally, or alternatively, an attribute associated with a given medical data sink may be used by the medical room control system to configure a medical data source. For example, the medical room control system can control the settings of a medical data source (e.g., a video source) so that the color space or resolution of the medical data from the medical data source is within the color space or resolution capability of the medical data sink to which the medical data is routed. By automatically configuring a medical data source or a medical data sink, the connection of a medical data source or medical data sink within a medical room environment may be simplified, reducing required set-up time and installation expertise.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like, refer to the action and processes of a computer system, or similar electronic computing device, that manipulates and transforms data represented as physical (electronic) quantities within the computer system memories or registers or other such information storage, transmission, or display devices.

The present disclosure, in some aspects, also relates to devices or systems for performing the operations herein. The devices or systems may be specially constructed for the required purposes, may comprise a general-purpose computer selectively activated or reconfigured by a computer program stored in the computer, or may include any combination thereof. Computer instructions for performing the operations herein can be stored in any combination of non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMS, EEPROMs, magnetic or optical cards, or any type of media suitable for storing electronic instructions, and each coupled to a computer system bus. One or more instructions for performing the operations herein may be implemented in or executed by one or more Application Specific Integrated Circuits (ASICs), Field Programmable Gate Arrays (FPGAs), Digital Signal Processing units (DSPs), Graphics Processing Units (GPUs), or Central Processing Units (CPUs). Furthermore, the computers referred to herein may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

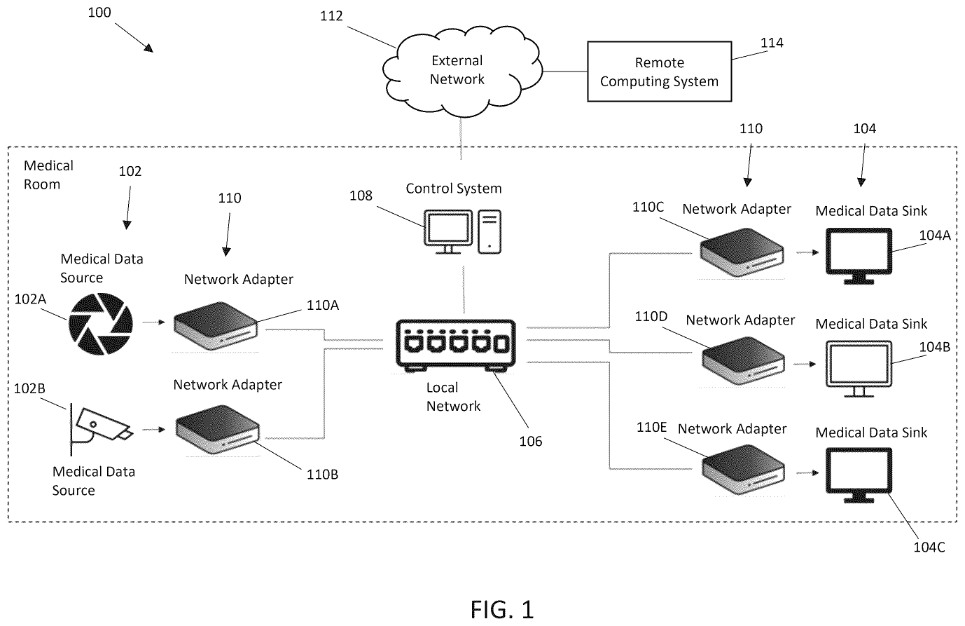

FIG. 1 illustrates an exemplary system 100. System 100 may operate in a medical room environment (e.g., a single medical room, a set of medical rooms, associated areas such as nurse's stations, etc.) and may include various devices that operate during a medical procedure. Some of the devices may generate medical data, in which case the devices are medical data sources 102 (such as a medical imaging system or component thereof). Some of the devices may use medical data received from another device, in which case the devices are medical data sinks 104 (such as a display or component thereof). Some devices may both generate and receive medical data, in which case the device is, or includes, both a medical data source 102 and a medical data sink 104. The various devices may be connected to a medical network 106 such that different kinds of data (such as video data, image data, audio data, measurement data, control data, or any other data) from the medical data sources 102 may be transmitted to the medical data sinks 104. The medical network 106 may be any suitable type of network or combination of types of networks. Optionally, the medical network 106 may be limited in range to cover a specific area, such as only one medical room, and the medical network 106 may be any appropriate network with a limited range, such as a local area network (LAN), virtual local area network (VLAN), or a Wireless Fidelity (WiFi) network. The medical network 106 may be connected to one or more external networks 112 for communication with one or more remote computing systems 114. For example, the medical network 106 may be a local area network specific to the medical room but may include a connection to a hospital network so that one or more devices within the medical room can communicate with one or more devices external to the medical room (such as a hospital information system) and/or may include a connection to a cloud network for accessing cloud storage and/or cloud computing.

A medical data source 102 may be any medical device or component of a medical device that generates data in the medical room environment, such as a medical imaging system or component thereof (for example, a C-arm fluoroscopic imager, an open-field imager, or an endoscopic imager), or may be any medical device that includes components for generating data, such as a surgical light that includes an in-light camera. As used herein, a “medical data source” is any device that is present in the medical room environment that generates data that is transmitted through the medical network, and “medical data” is any data generated by a medical data source. Thus, for example, a medical data source 102 may be a pan-tilt-zoom (PTZ) camera used for observation of the medical room environment but not specifically for providing medical care. While a PTZ camera may be used to generate medically relevant data (data that may be used in providing medical treatment), it may also be used to generate non-medically relevant data, such as a video stream of the medical room. A medical data source 102 may be a keyboard, video, and mouse (KVM) source, such as a workstation in the medical room that allows users (e.g., surgeons, medical staff, and the like) to control the various other devices in the medical room for a variety of purposes. For example, a camera may lose focus, but instead of a user directly adjusting the focus, which may compromise sterilization, the user may adjust the focus remotely through the KVM source. As such, data generated by a medical data source 102 may include command data.

Each medical data source 102 may be connected to a respective network adapter 110 that encodes data generated by the medical data source 102 to a format that can be transmitted through the medical network 106. The format that the network adapter 110 encodes the data to may be any appropriate format that allows the encoded data to be transmitted through the medical network. For example, the network adapter 110 may encode the data to a series of packets that each include additional network transmission metadata. Specifically, the network adapter 110 may encode the data to a packetized form according to the communication protocol used by the medical network 106, such as an Internet Protocol (IP) (e.g., User Datagram protocol (UDP) or Transmission Control protocol (TCP)). In such cases, the network adapter 110 may encode the data generated by the medical data source 102 to IP packets that are then transmitted through the medical network 106. Medical data sources 102 and medical data sinks 104 may be configured to generate and use, respectively, video data, and network adapters 110 may be video network adapters.

A network adapter 110 may be used specifically for a particular medical data source 102. For example, medical data source 102A may be a PTZ camera, and network adapter 110A may be used specifically for the PTZ camera. A medical data source 102 and network adapter 110 may be used together as part of a mobile setup. For example, medical data source 102B may be an endoscopic imaging system carried by a mobile cart, and network adapter 110B may be connected to the endoscopic imaging system and carried by the mobile cart. The mobile cart may be moved between different medical rooms depending on where it is needed. A network adapter 110 may be useable with multiple different medical data sources 102 depending on which medical data source 102 is connected to the network adapter 110. For example, a network adapter 110 may be built into (or configured as) a wall plate (or other type of connection plate) to which one or more medical data sources 102 may be connected at various times. For example, a mobile endoscopic imaging system may be moved into a particular medical room during set-up of the room in preparation for a given medical procedure, and the mobile endoscopic imaging system may be connected to a wall plate having a network adapter 110.

While a medical data source 102 may be connected to a network adapter 110 that is a separate component from the medical data source 102, a medical data source 102 may have an integrated network adapter 110 that is a component of the medical data source 102. As a result, conversion of data to a packetized format for transmission through the medical network 106 may be performed by the integrated network adapter 110 of the medical data source 102.

A network adapter 110 may include any suitable network interface for transmitting data through the medical network 106. The network interface may be any appropriate component that is able to establish a connection to the medical network 106, which may depend on the type of network that is used for medical network 106. For example, if the medical network 106 is a wired network, the network interface may be configured to transmit data on the wired network. Alternatively, if the medical network 106 is a WiFi network, the network interface may include a transceiver that generates and receives radio waves.

Medical data sinks 104 may include any device capable of using data generated by a medical data source 102. For example, the medical data sinks 104 may include one or more displays for displaying various types of data, such as user interfaces, images, and/or video feeds generated by one or more of the medical data sources 102. Other examples of medical data sinks 104 include, but are not limited to, a data recorder, a speaker, a printer, a projector, a headset, or any remotely controllable device. The medical data sinks 104 may be configured to process data in a native format, which may correspond to the native format of the data generated by a given medical data source 102 or may be a different native format, but in either case, may differ from the packetized format that is used when transmitting data through the medical network 106. As such, each medical data source 102 may be connected to a network adapter 110 that decodes the packetized data from the medical network 106 to the format supported by the given medical data sink 104. The network adapter 110 may be a separate component from the medical data sink 104 or may be an integrated component of the medical data sink 104.

System 100 includes a medical room control system 108 that may control the generation, transmission, and/or use of medical data by the medical data sources 102 and medical data sinks 104. Medical room control system 108 may control routing through the network 106 such that medical data from a given medical data source 102 is routed to at least one particular medical data sink 104. For example, medical room control system 108 may control routing through the network 106 such that video from medical data source 102A is routed to network adapter 110C for use by medical data sink 104A, and video from medical data source 102B is routed to network adapter 110D for use by medical data sink 104B (and/or to network adapter 110E for use by medical data sink 104C).

Medical room control system 108 may generate and use medical data transmitted via the network 106 and, thus, may be both a medical data source and a medical data sink. Medical room control system 108 may include one or more network adapters 110 and/or may include network adapter functionality to decode medical data from a packetized format to a format supported by the medical room control system 108 and encode medical data to a packetized format for transmission over the network 106. Medical room control system 108 may be configured to process medical data received from a medical data source 102, such as by increasing the resolution of image or video data, and transmit the processed medical data to a medical data sink 104. For example, medical data source 102A may generate video data that is encoded by network adapter 110A and transmitted through the medical network 106. That medical data may then be received at medical room control system 108, which converts the medical data to a native format for processing. The medical room control system 108 may then process the video data, encode the processed video data to a packetized format, and transmit the processed medical data through the network 106. The processed medical data may then be received by a medical data sink 104A (e.g., a display) via network adapter 110C.

Medical room control system 108 may control the generating of medical data by a medical data source 102 and/or the use of medical data by a medical data sink 104 based on attributes of the medical data source 102 and/or medical data sink 104. For example, medical room control system 108 may control settings of medical data source 102A based on one or more attributes of medical data sink 104A such that medical data transmitted from medical data source 102A to medical data sink 104A is configured for use by medical data sink 104A. Similarly, medical room control system 108 may control settings of medical data sink 104A based on one or more attributes of medical data source 102A such medical data sink 104A uses (i.e., processes) medical data from medical data source 102A in a manner suitable to the medical data.

The attributes of a medical data source 102 and/or medical data sink 104 that may be used by medical room control system 108 for controlling the generating of medical data by a medical data source 102 and/or the use of medical data by a medical data sink 104 may be determined by the medical room control system 108 based on information stored by a network adapter 110 that is associated with the medical data source 102 or the medical data sink 104. Medical room control system 108 may obtain information related to attributes of the medical data sources 102 and medical data sinks 104 from the various network adapters 110 and may use (directly or indirectly) the information to control the generating of medical data by a medical data source 102 and/or the use of medical data by a medical data sink 104.

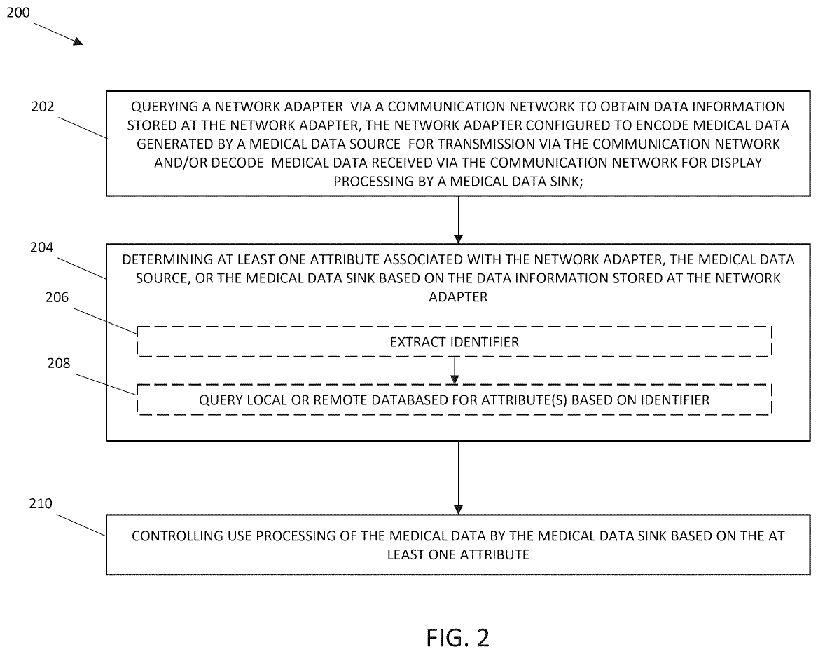

FIG. 2 illustrates an exemplary method 200 for controlling use of medical data by one or more medical data sinks based on information associated with one or more medical data sources, medical data sinks, and/or one or more network adapters that is stored at the one or more network adapters. Method 200 may be used, for example, during set-up of one or more of the medical data sources, medical data sinks, and/or network adapters, such as during installation of medical data sources and sinks in a medical room. Method 200 may facilitate the configuring of connections between medical data sources and medical data sinks and/or the configuring of medical data sources and/or medical data sinks, which may reduce the time and expense required for set-up of a medical room and/or may reduce the number of errors arising during a set-up. Method 200 may be used during an initial set-up of a medical room and/or at any time that a new or different medical data source and/or medical data sink is added to the medical room.

One or more steps of method 200 may be performed by a computing system connected via a network to one or more medical data sources and/or one or more medical data sinks. For example, one or more steps of method 200 may be performed by medical room control system 108 of FIG. 1.

At step 202, the medical room control system queries at least one network adapter via a communication network to obtain information stored at the at least one network adapter. A network adapter that is queried can be associated with a particular medical data source for encoding medical data generated by the medical data source for transmission via the communication network. A network adapter that is queried may be associated with a particular medical data sink for decoding medical data received via the communication network for processing by a medical data sink. For example, with reference to FIG. 1, the at least one network adapter queried at step 202 can include network adapter 110A associated with medical data source 102A, network adapter 110B associated with medical data source 102B, network adapter 110C associated with medical data sink 104A, and/or network adapter 110D associated with medical data sink 104B.

Step 202 can include querying as few as one network adapter connected to the communication network and as many as all network adapters connected to the communication network. Step 202 can include querying a single network adapter at a time or can include broadcasting a query message to all network adapters connected to the network. Step 202 can include or be a part of a discovery routine in which the medical room control system discovers one or more of the network adapters that are connected to the communication network and establishes communication with the one or more network adapters. For example, step 202 can be performed upon connection of a network adapter to the communication network (e.g., once the network adapter has been powered on) and can be repeated for any additional network adapter that becomes connected to the communication network. Step 202 can be performed upon connection of the medical room control system to the communication network (e.g., physical connection or power up of the medical room control system or communication network) and/or at a predefined step in a medical procedure (e.g., during a set-up process performed in preparation for a surgical procedure). Step 202 generally includes transmitting a request to one or more network adapters to provide the sought-after information stored at the at least one network adapter. Each network adapter that receives the request may respond by transmitting the sought-after information to the medical room control system.

The information stored at a given network adapter that is queried by the medical room control system at step 202 may be associated with the medical data source or medical data sink with which the network adapter is associated and/or may be associated with the network adapter itself. The information can include one or more identifiers associated with the medical data source or sink and/or network adapter, such as an identifier specific to the medical data source or sink or the network adapter (e.g., a serial number) and/or an identifier specific to a type of the medical data source or sink or the network adapter, such as a model number or Global Trade Item Number (GTIN). For example, the information stored at network adapter 110A of FIG. 1 may include the GTIN of medical data source 102A, and the information stored at network adapter 110C may include the GTIN of medical data sink 104A. Optionally, the identifier can include a combination of a unique identifier for the particular medical data source or sink or the network adapter and a device type identifier. For example, the identifier can be a unique device identifier (UDI), which includes a device identifier (such as a GTIN) and a product identifier (such as a serial number).

The identifier may correspond to a particular role of the medical data source or sink or the network adapter in a particular system. Such an identifier may be a “friendly name” assigned to the given medical data source or sink according to the role of the medical data source or sink in the system. For example, with reference to FIG. 1, medical data sink 104A may be a display mounted to a boom, and medical data sink 104B may be a display of a mobile cart, and the identifier stored at network adapter 110C may be “Boom-Mounted Display,” and the identifier stored at network adapter 110D may be “Mobile Cart Display.”

Additionally, or alternatively, the information stored at a network adapter that is queried at step 202 can include at least one attribute associated with the network adapter and/or the corresponding medical data source or sink. For example, medical data source 102A may be a surgical camera, and the information stored at network adapter 110A may include a resolution, color space, and/or other attribute of video data generated by surgical camera. Similarly, medical data sink 104A may be a display, and the information stored at network adapter 110C may include a resolution, color space, and/or other attribute of the display.

Attributes associated with the network adapter and/or the corresponding medical data source or sink may include software and/or firmware version and/or data associated with the age of the network adapter and/or the medical data source or sink, such as installation date, device uptime, or number of device uses. Such attributes may facilitate system and/or device maintenance or replacement.

Attributes associated with a network adapter may include (additionally or alternatively to any of the attributes discussed above) attributes corresponding to the communication capability of the network adapter, such as communication protocol, a data rate, and/or a connection type (e.g., fiber or Ethernet connection). Attributes associated with a network adapter may include one or more attributes associated with performance of the network adapter, such as a communication power of the network adapter and/or a signal-to-noise ratio of the connection of the network adapter to the network.

At step 204, the medical room control system may determine at least one attribute associated with the network adapter and/or the associated medical data source or sink based on the information stored at the network adapter. For example, medical room control system 108 of FIG. 1 may determine at least one attribute of network adapter 110A and/or at least one attribute of medical data source 102A based on information stored at network adapter 110A that network adapter 110A provided to medical room control system 108 in response to a query sent by medical room control system at step 202. The medical room control system may determine at least one attribute associated with multiple different devices, such as multiple different network adapters, medical data sources, and/or medical data sinks. For example, medical room control system 108 may receive responses to its information query from each of the network adapters 110 and may use the information to determine at least one attribute associated with each network adapter 110, each medical data source 102, and/or each medical data sink 104 of system 100.

The medical room control system may extract at least one attribute from the information received from a given network adapter. For example, as discussed above, the information stored at a given network adapter may include at least one attribute of the network adapter and/or at least one attribute of the associated medical data source or sink, and the network adapter may respond to the query of the medical room control system at step 202 by providing the at least one attribute in the reply to the medical room control system. The medical room control system may parse the reply to extract the at least one attribute associated with the network adapter and/or the medical data source or sink associated with the network adapter.

In some variations, one or more attributes determined by the medical room control system at step 204 may not be included in the information provided by the network adapter but may be stored in a memory of the medical room control system and/or hosted by a remote computing system. The information provided by a network adapter to the medical room control system may include an identifier associated with the network adapter and/or the associated medical data source or sink, and step 204 may include optional step 206 of extracting the identifier from the information received from the network adapter and optional step 208 of querying a database of attributes based on the identifier to obtain at least one attribute.

The database may be stored locally in a memory of the medical room control system and/or may be hosted remotely (e.g., stored in a memory of remote computing system 114 of FIG. 1). A database of attributes may associate attributes with an identifier corresponding to a device having one or more attributes, and the medical room control system may use a database query that includes the identifier to obtain the one or more attributes associated with the identifier. Any of the attributes discussed herein can be stored in a database of the medical room control system and/or a remote computing system. Storing attributes at the medical room control system and/or at a remote computing system may reduce the amount of information that a network adapter is required to store and, thereby, the amount of memory it is required to have. Storing attributes at the medical room control system and/or at a remote computing system may provide the additional advantage of enabling the attributes to be updated more easily should there be a change in the attributes of a network adapter, medical data source, and/or medical data sink. For example, a change to the attributes of a model of a medical data sink (e.g., a model of a display) resulting from a firmware update to the model may be implemented simply by updating an entry associated with the model (or entries of the medical data sources of the model) in a database, rather than by updating each network adapter associated with a medical data source of the model.

Step 204 may include the medical room control system using an identifier that is specific to a particular device, such a serial number, to obtain attributes specific to the particular device from a local or remote database, such as one or more attributes associated with age or use of a given device (e.g., install date, number of uses, device uptime, etc.). Alternatively, step 204 may include the medical room control system using an identifier that is specific to a type of a particular device, such as a GTIN, to obtain attributes specific to the particular type of device (such as capabilities common to the model of the particular device) from a local or remote database.

The attribute(s) associated with a network adapter, a medical data source, or a medical data sink that is determined in step 204 may include an association of a medical data source with a medical data sink. For example, with reference to FIG. 1, an attribute determined by medical room control system 108 based on information received from network adapter 110A may be an association between medical data source 102A and medical data sink 104A, indicating, for example, that medical data generated by medical data source 102A should be routed to medical data sink 104A. The association may include “friendly names.” For example, an association comprised of “Surgical Light Camera” and “Boom-Mounted Display” may indicate that the medical data generated by a medical data source connected to the network adapter that provided the information from which the associated was determined should be routed to the network adapter associated with the “Boom-Mounted Display” (which may be determined at step 204 based on information from the network adapter associated with that medical data sink).

Optionally, an association of a medical data source with a medical data sink may be a fixed association, such as an association that is defined during manufacturing and/or installation and that generally does not change. In some examples, however, the association is dynamic and can be updated automatically. For example, the association (stored locally on the network adapter, locally on the medical room control system, or hosted by a remote database) may be updated based on how medical data from a medical data source has been routed in previous set-ups. For example, a user may manually set the routing of a mobile imaging system within a medical room so that the medical data from the mobile imaging system is routed to a particular display within the medical room, and an attribute associated with the mobile imaging system (e.g., stored on the network adapter associated with the mobile imaging system or stored in an attribute database of the medical room control system or a remote computing system) may be updated to reflect an association of the mobile imaging system with the particular display so that the mobile imaging system is automatically routed to that display the next time the mobile imaging system is connected to the network of the medical room (e.g., after returning from being used in another medical room).

At step 210, the medical room control system may control use of the medical data by a medical data sink based on the at least one attribute determined at step 204. Controlling use of the medical data by a medical data sink may include configuring the medical data sink and/or configuring the medical data source that generates the medical data (e.g., so that the medical data does not need to be modified by the medical data sink). For example, controlling use of the medical data by a medical data sink may include configuring the medical data sink by sending an instruction to the medical data sink to adjust at least one setting of a medical data sink based on an attribute of a medical data source that generated the medical data. For example, with reference to FIG. 1, medical data sink 104A may be a display that is used within system 100 to display video from medical data source 102A (e.g., a camera), and medical room control system 108 may send an instruction to medical data sink 104A to adjust its resolution and/or color settings based on a resolution attribute and/or color setting attribute of the medical data source 102A determined at step 204 based on information received from network adapter 110A at step 202.

Controlling use of the medical data by a medical data sink, according to step 210, may include configuring the medical data source that generates the medical data by adjusting at least one setting of the medical data source according to at least one attribute of the medical data sink. For example, continuing with the example arrangement above in which medical data sink 104A displays video from medical data source 102A, medical room control system 108 may send an instruction to medical data source 102A to adjust its resolution and/or color space settings based on a resolution attribute and/or color space attribute of the medical data sink 104A determined at step 204 based on information received from network adapter 110C at step 202.

Controlling use of the medical data by a medical data sink, according to step 210, may include the medical room control system modifying the medical data generated by a medical data source and providing the modified medical data to the medical data sink. For example, medical room control system 108 may adjust a resolution and/or a color space of video received from medical data source 102A based on a resolution attribute and/or a color space attribute of the medical data sink 104A determined at step 204 based on information received from network adapter 110C at step 202 (and/or based on a resolution attribute and/or color space attribute of the medical data source 102A determined at step 204 based on information received from network adapter 110A at step 202).

Controlling use of the medical data by a medical data sink, according to step 210, may include controlling routing of the medical data from the medical data source that generated it to the medical data sink. For example, medical room control system 108 may receive a friendly name from network adapter 110A that is associated with medical data source 102A (e.g., “Endoscopic Camera”) and a friendly name from network adapter 110C that is associated with medical data sink 104A (e.g., “Primary Display”) and may query a database (local or remote) to determine the medical data sink to which the medical data from Endoscopic Camera should be routed and the medical data source from which the medical data for the Primary Display should be routed. The database may include an association of Endoscopic Camera with Primary Display indicating that medical data from the Endoscopic Camera should be routed to the Primary Display. Based on this association, medical room control system 108 may update a routing table (its own routing table or the routing table of one or more routers of network 106) so that medical data transmitted by network adapter 110A is routed to network adapter 110C.

Controlling use of the medical data by a medical data sink, according to step 210, may include controlling routing based on network adapter associations. For example, an attribute database may include an association between network adapter 110A and network adapter 110C (e.g., between a unique identifier or friendly name for network adapter 110A and a unique identifier or friendly name for network adapter 110C) indicating that medical data transmitted by network adapter 110A should be routed to network adapter 110C. This may be useful, for example, where a given network adapter can be connected to by different medical data sources (e.g., the network adapter is integrated into a wall plate to which medical data sources can be selectively connected), but regardless of which medical data source is connected, the medical data should be routed to a particular network adapter or medical data sink. For example, a medical room may include a wall plate that includes network adapter 110A and that is designated for connecting mobile endoscopic camera systems (e.g., the wall plate may include a suitable label indicating that the wall plate is intended for connecting a mobile endoscopic camera system). The particular mobile endoscopic camera system that is connected to the wall plate may differ from time to time (e.g., mobile endoscopic camera systems may be moved from medical room to medical room depending on type of procedure and availability), but the medical room may be configured such that the video feed from whatever specific mobile endoscopic camera system is connected to the wall plate should be displayed on a display connected to a particular network adapter. As such, routing may be based on an identifier for the network adapter 110A, rather than for medical data source 102A.

Method 200 may be performed automatically by medical room control system such that medical data from a medical data source can be automatically handled with as little user effort as connecting the medical data source to the network and powering it on. Optionally, one or more steps of method 200 may be accompanied by one or more user prompts that enable a user to verify and/or change decision points. For example, method 200 may include requesting user confirmation of an attribute obtained in step 204 or user confirmation of a particular routing between a medical data source and a medical data sink in step 206. A notification that includes a user confirmation may be displayed to a user, such as via a display of the medical room control system.

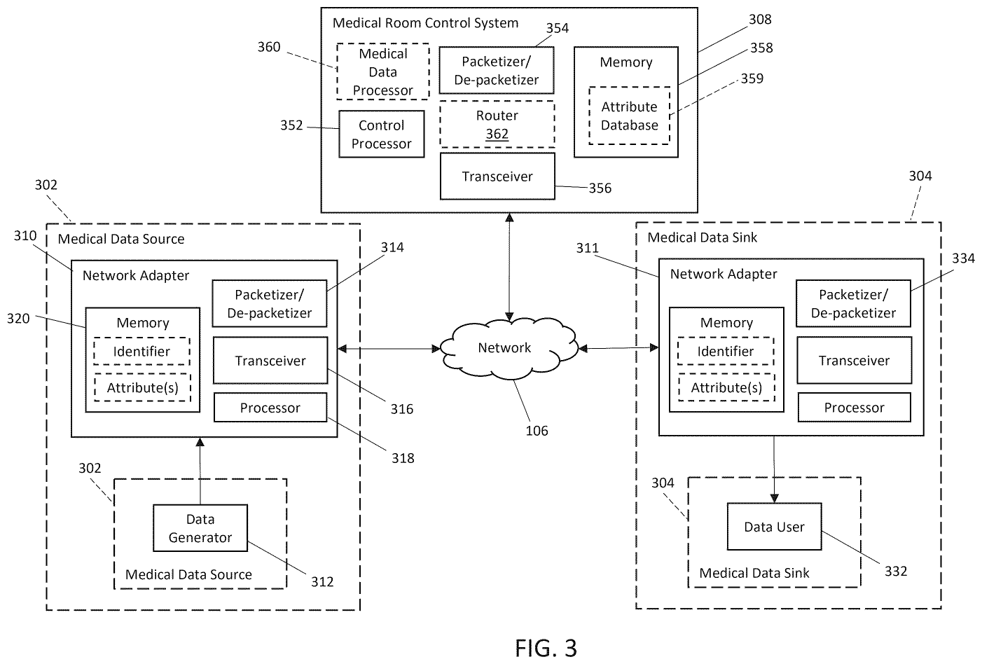

FIG. 3 is a functional block diagram of examples of medical data source 102, medical data sink 104, network adapter 110, and medical room control system 108 of system 100. The functional blocks of FIG. 3 reflect the functional capabilities of the respective devices and may be implemented by any combination of one or more general purpose processors, one or more special purpose processors, dedicated circuitry, and/or software components. As such, the functional blocks of FIG. 3 are not intended to imply discrete hardware components or discrete software components, but rather, may be implemented using any suitable combination of software and hardware.

Medical data source 302 (which may be used for one or more of the medical data sources 102 of system 100) includes a data generator 312, which in general terms is responsible for generating the medical data that is provided by medical data source 302. For example, medical data source 302 may be a camera system, and the data generator 312 includes the components of the camera system that generate images or video frames. The data generator 312 is communicatively connected to a network adapter 310 (which may be used for one or more network adapters 110 of system 100). The medical data source 302 and the network adapter 310 may be separate components that are connected via, for example, one or more cables, or the medical data source 302 may include the network adapter 310. In either arrangement, the network adapter 310 receives medical data generated by the data generator 312.

The network adapter 310 may include a packetizer/de-packetizer 314 that converts (encodes) medical data from data generator 312 into packets that can be transmitted to the network 106 by transceiver 316 and de-packetizes packets received by transceiver 316 via the network 106. The packetizer/de-packetizer 314 may be configured to convert different types of data such that the network adapter 310 may be used for different types of medical data sources 302 and/or different types of medical data (e.g., network adapter 310 may include multiple different types of data connectors, such as an HDMI connector, a DisplayPort connector, an SDI connector, and/or a USB-C connector). The packetizer/de-packetizer 314 may be configured specifically for use with a particular type of medical data source 302. In addition to encoding data, the packetizer/de-packetizer 314 may convert (decode) data received from the network 106 via transceiver 316, such as control data received from medical room control system 308 (which may be used for medical room control system 108 of system 100).

Network adapter 310 includes a processor 318 that controls the functionality of the network adapter 310 and may handle communications with medical room control system 308 and/or one or more other network adapters. Network adapter 310 includes memory 320 that may store information associated with the network adapter 310 and/or information associated with medical data source 302. As described above, the information may include one or more identifiers and/or one or more attributes associated with the medical data source 302 and/or the network adapter 310. Processor 318 may process a query received from medical room control system 308, according to step 202 of method 200, by obtaining information associated with the network adapter 310 and/or information associated with medical data source 302 from memory 320 and sending the information to medical room control system 308.

Information stored in memory 320 may be loaded onto memory 320 during manufacture. For example, an identifier of the network adapter 310 may be loaded onto memory 320 during manufacture of the network adapter 310. For example, an identifier of the medical data source 302 (e.g., a GTIN and/or a friendly name) and/or one or more attributes of the medical data source 302 may be loaded onto memory 320 prior to shipment of the network adapter 310 to a customer (e.g., based on an order form indicating the medical data source 302 will be used with the network adapter 310). Information stored in memory 320 may be loaded onto memory 320 during installation at a medical facility (e.g., during installation in a medical room). For example, an identifier of a medical data source (e.g., a GTIN and/or a friendly name) may be loaded onto memory 320 during set-up of a medical room based on which medical data source is connected to memory 320. Information stored in memory 320 may be updated over time. For example, an attribute of medical data source 302 that is stored in memory 320 may be updated after a firmware update of the medical data source 302. The attribute may be updated, for example, by a field service engineer connecting a flash drive or computing system directly to the network adapter 310 and/or by instructions provided by medical room control system 308 via network 106.

Optionally, information stored in memory 320 may be updated by the processor 318. For example, the processor 318 may monitor various aspects of the operation of the network adapter 310 and may maintain and update information stored in memory 320 accordingly. Examples of aspects of the operation of the network adapter 310 that may be monitored and recorded in memory 320 include, but are not limited to, the uptime (e.g., the current power-on clock time and/or the lifetime power-on clock time) of the network adapter 310 and/or medical data source 302, the number of uses of the network adapter 310 and/or medical data source 302 (e.g., based on the number of power cycles or the number of medical data transmissions), and a signal-to-noise ratio of the connection of the network adapter 310 to the network 106.

Medical data sink 304 includes a data user 332, which in general terms is responsible for using the medical data that is received via the network 106. For example, the medical data sink 304 may be a display, and the data user 332 includes the components of the display that process digital images or video and outputs the digital images or video on a display screen. The data user 332 is communicatively connected to a network adapter 311 (which may be used for one or more network adapters 110 of FIG. 1). The medical data sink 304 and the network adapter 311 may be separate components that are connected via, for example, one or more cables, or the medical data sink 304 may include the network adapter 311. In either arrangement, the network adapter 311 provides medical data to the data user 332.

Network adapter 311 may include similar functionality as network adapter 310. In some variations, network adapter 310 and network adapter 311 are the same type of network adapter. In some variations, network adapter 310 is configured for connection to one or more types of medical data sources, whereas network adapter 311 is configured for use with one or more types of medical data sinks. For brevity, details of functionality of network adapter 311 that may be the same as network adapter 310 are not repeated.

Packetizer/de-packetizer 334 may be configured for assembling packets received via network 106 into medical data that is in a form usable by data user 332. Packetizer/de-packetizer 334 may be configured for assembling packets for use by various different types of medical data sources 302 and/or different types of medical data (e.g., network adapter 311 may include multiple different types of data connectors, such as an HDMI connector and a USB-C connector, and the de-packetizer may be configured to convert packets into medical data according to an HDMI data format or a USB-C data format). Packetizer/de-packetizer 334 may be configured for converting data into packets for transmission via network 106. For example, packetizer/de-packetizer 334 may convert a communication directed to medical room control system 308 (e.g., a response to an information query according to step 202 of method 200) into packets for transmission over network 106.

Medical room control system 308 includes a control processor 352 configured to communicate with network adapters 310 and 311, such as for obtaining information associated with the network adapters 310 and 311, the medical data source 302, and/or the medical data sink 304, and/or for controlling the network adapters 310 and 311, the medical data source 302, and/or the medical data sink 304 (e.g., configuring the network adapters 310 and 311, the medical data source 302, and/or the medical data sink 304). Medical room control system 308 includes a packetizer/de-packetizer 354 for converting packets received via the network 106 by transceiver 356 into data usable by the medical room control system 308 and converting data into packets for transmission by transceiver 356 over the network 106. Medical room control system 308 may include a router 362 that may handle routing of data between network adapters connected to network 106. Optionally, router 362 or its functionality is provided by one or more routers of network 106 (e.g., network 106 includes one or more routers), and medical room control system 308 may be configured to control the one or more routers of network 106, such as to update a routing table.

Medical room control system 308 may include a memory 358, which may store an attribute database 359 that the control processor 352 may query to obtain attributes associated with the network adapters 310 and 311, the medical data source 302, and/or the medical data sink 304 (e.g., using identifiers). Optionally, memory 358 may store some attributes and a remote storage (e.g., remote computing system 114 shown in FIG. 1) may store other attributes. In some variations, memory 358 does not store any attributes (e.g., attributes are stored at the network adapters 310 and 311 and/or in a remote database). Memory 358 may store user profiles that medical room control system 308 may use to configure medical data source 302, medical data sink 304, processing of medical data from medical data source 302, routing of medical data, or any other aspect of medical data handling described herein. Memory 358 may store any other information usable by the medical room control system 308 for performing the functionality described herein, including the steps of method 200 of FIG. 2.

Medical room control system 308 may include (or be operatively connected to) one or more medical data processors 360 that may modify medical data received from one or more medical data sources. For example, medical data source 302 may be a camera system, and the medical data processor 360 may modify video received from medical data source 302 to adjust a resolution and/or color space of the video data prior to sending the medical data to medical data sink 304, with the adjustment to the resolution and/or color space being based on one or more attributes of the medical data sink 304. Additionally, or alternatively, the medical data processor 360 may overlay information related to the medical data source 302 onto the video prior to sending the video to the medical data sink 304.

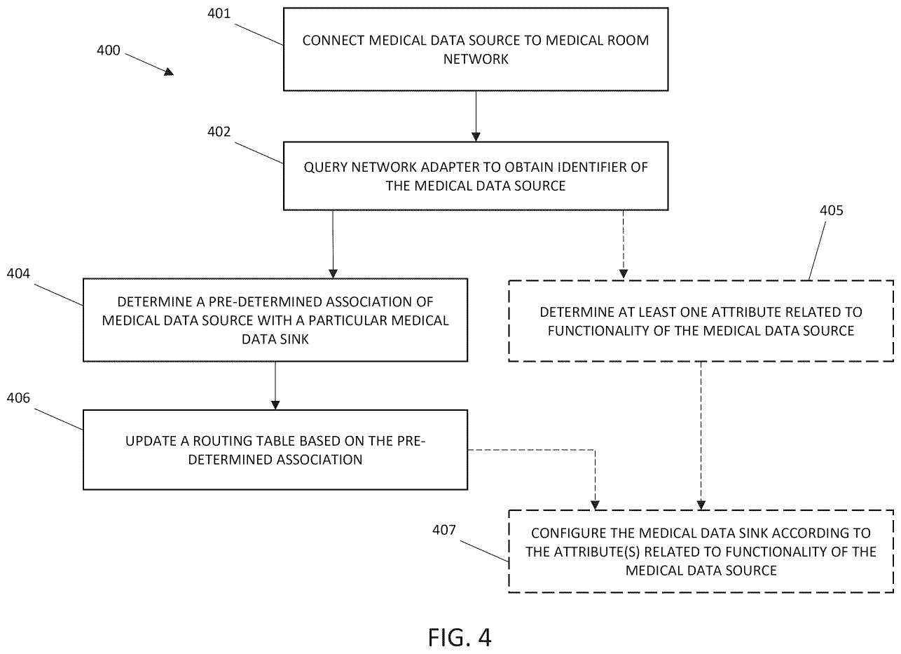

As noted above, method 200 can be used during the installation of one or more medical data sources or medical data sinks, such as when a new medical room is being configured or when a new device is being added to the medical room. FIG. 4 illustrates an example implementation of method 200 for adding a medical data source to a system, such as adding any of the medical data sources 102 and medical data sinks 104 to system 100 of FIG. 1. Method 400 can be used to connect a new medical data source or sink to the system, such as when installing medical data sources or sinks in a new medical room. Method 400 can be used to reconnect a medical data source or sink to the system, such as to reconnect a mobile medical data source (e.g., an endoscopic imaging cart) or mobile medical data sink that is moved into a medical room. Method 400 is described below with reference to medical data source 102A and network adapter 110A but can be used for any medical data source or medical data sink.

At step 401, a user (e.g., an installer or medical personnel) connects medical data source 102A to network 106. The medical data source 102A may be connected to network 106 by using a network cable (e.g., an Ethernet cable or fiber optic cable) to connect network adapter 110A to a cable connector of network 106 (an Ethernet connector of a wall plate) and powering on the medical data source 102A and network adapter 110A. For example, medical data source 102A and network adapter 110A may be pre-connected (e.g., connected “out of the box” from the manufacturer or components of a mobile medical device), or the network adapter 110A may be integrated into the same device as medical data source 102A. Alternatively, the medical data source 102A may be connected to network 106 by using a cable (e.g., a cable such as an HDMI cable or a DP cable, or a network cable such as an Ethernet cable or fiber optic cable) to connect medical data source 102A to network adapter 110A, which is already connected to the network 106, and powering on medical data source 102A. For example, network adapter 110A may be configured as a wall plate (or included in a wall plate) to which medical data source 102A is connected.

At step 402, the medical room control system 108 queries network adapter 110A via network 106 to obtain at least on identifier of the medical data source 102A that is stored at the network adapter 110A, according to an example of step 202 of method 200 of FIG. 2. Network adapter 110A receives and responds to the query with its stored identifier for medical data source 102A. The identifier may be, for example, a GTIN or friendly name of the medical data source 102A. The identifier(s) may have been pre-loaded onto the network adapter 110A prior to connecting the network adapter 110A to the network 106. For example, a manufacturer that provides the medical data source 102A and the network adapter 110A to a customer may have stored the identifier for medical data source 102A on the network adapter 110A prior to shipping to the customer. For example, a friendly name for the medical data source 102A may have been stored in the network adapter 110A prior to shipping to the customer, along with indicators (e.g., labels) that the medical data source 102A and network adapter 110A should be connected to one another (and/or where they should be positioned in the medical room for which they are destined).

At step 404, the medical room control system 108 may determine a pre-determined association of medical data source 102A with a particular medical data sink based on the identifier obtained from network adapter 110A, in accordance with an example of step 204 of method 200. For example, the medical room control system 108 may determine a pre-determined association of medical data source 102A with medical data sink 104B of system 100, indicating that medical data generated by medical data source 102A should be routed to medical data sink 104B. Step 404 may include the medical room control system 108 extracting the identifier(s) from the information provided by the network adapter 110A and querying an attribute database (a local database or a remotely hosted database) based on the identifier(s). For example, the identifier may include a friendly name of medical data source 102A (e.g., “Endoscopic Imager”), and the database may include an entry that associates Endoscopic Imager with a friendly name of a medical data sink 104B (e.g., “Display 2”). The pre-determined association of medical data source with a particular medical data sink may be specific to a user profile. For example, different users may have different routing preferences and an attribute database may include different attributes (e.g., different associations of medical data sources with medical data sinks) for different user profiles. Additionally, or alternatively, the pre-determined association of medical data source with a particular medical data sink may be specific to a particular type of medical room such that similar medical rooms are set-up in similar ways. As such, step 404 may include medical room control system 108 determining a pre-determined association of medical data source 102A with a particular medical data sink based on the identifier obtained from network adapter 110A and based on a type of medical room setup (e.g., a minimally invasive surgery room versus a general surgery room).

At step 406, the medical room control system 108 may update a routing table based on the pre-determined association determined at step 404, according to an example of step 210 of method 200. For example, medical room control system 108 may update a routing table (e.g., its own routing table or a routing table of a router of the network 106) so that medical data transmitted from network adapter 110A (which is connected to medical data source 102A) over network 106 is routed to the network adapter 110D (which is connected to medical data sink 104B). Thus, the medical data source 102A can be automatically connected to the network 106 and its medical data routed appropriately without requiring an installer to manually assign the medical data source 102A to a medical data sink. Thus, method 400 may reduce installation time and enable non-experts to add medical data sources and/or sinks to a given medical room. Additionally, or alternatively, method 400 may allow for easier movement of mobile medical data sources and/or medical data sinks between medical rooms.

Optionally, method 400 may include optional step 405 in which the medical room control system determines at least one attribute related to functionality of the medical data source 102A based on the identifier obtained from network adapter 110A. For example, medical room control system 108 may query an attribute database using a GTIN or serial number of the medical data source 102A to retrieve the attribute(s) related to functionality of the medical data source 102A.

At optional step 407, the medical room control system may configure the medical data sink 104B according to the attribute(s) related to functionality of the medical data source 102A. For example, the at least one attribute may be a resolution or color space of video generated by the medical data source 102A (obtained, for example, by querying the attribute database using the GTIN of the medical data source 102A), and medical room control system 108 may configure medical data sink 104B by adjusting a setting of the medical data sink 104B to use the resolution or color space when displaying the video. Alternatively, the medical room control system 108 may adjust the video data (which is routed through it) to change a resolution and/or color space of the video data according to a source resolution and/or color space (e.g., an attribute of medical data source 102A obtained using the identifier from network adapter 110A) and a sink resolution and/or color space (e.g., an attribute of medical data sink 104B obtained using an identifier from network adapter 110D). By automatically configuring a medical data source, a medical data sink, and/or the processing of medical data by the medical room control system, method 400 may facilitate set-up of a medical data source or medical data sink, reducing the set-up time and the expertise required by an installer.

Optionally, the use of the medical data generated by medical data source 102A may be controlled at step 406 based on a user profile. For example, different users (e.g., different surgeons) may have different preferences associated with which medical data sink is assigned to use the medical data from medical data source 102A. For example, the medical data source 102A may be an endoscopic camera, and different surgeons may prefer that the images or video generated by the endoscopic camera be displayed on different displays. A user profile may indicate such preference using friendly names associated with medical data sources and medical data sinks. For example, a first user profile may associate friendly name “Endoscopic Camera” with friendly name “Display 1,” and a second user profile may associate Endoscopic Camera with friendly name “Display 2.” Based on this association, a routing table may be updated during a set-up process for setting up for a medical procedure to be performed by the first user to route medical data from medical data source 102A to a medical data sink having the friendly name Display 1, and the routing table may be updated during a set-up process for setting up for a medical procedure to be performed by the second user to route medical data from medical data source 102A to a medical data sink having the friendly name Display 2. Additionally, or alternatively, one or more settings of a medical data source or a medical data sink may be configured according to a user profile. For example, a first user may prefer particular display brightness and/or contrast settings, and the medical room control system 108 may configure the display accordingly, such as during a set-up process for setting up for a medical procedure to be performed by the first user.

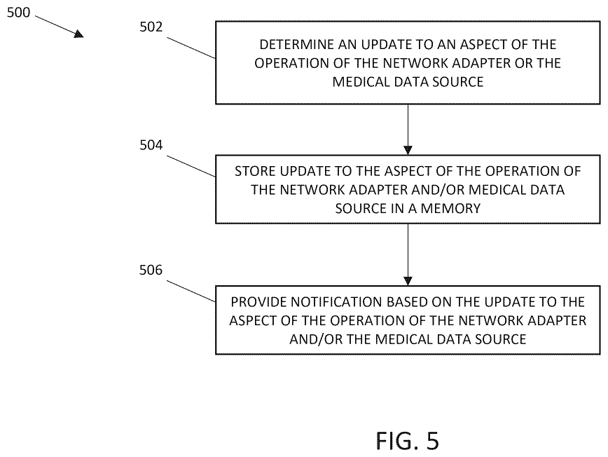

As mentioned above, a network adapter may be used to monitor one or more aspects of operation of a medical data source, a medical data sink, and/or a network adapter. FIG. 5 illustrates an exemplary method 500 for monitoring one or more aspects of the operation of a medical data source (e.g., medical data source 102), a medical data sink (e.g., medical data sink 104), and/or a network adapter (e.g., network adapter 110). Method 500 can be performed in addition to, or alternatively to, method 200 of FIG. 2 and method 400 of FIG. 4. Method 500 is described below with reference to monitoring one or more aspects of the operation of network adapter 110A and/or medical data source 102A, but it should be understood that any network adapter, medical data source, and/or medical data sink (and/or any combination of these) may be monitored according to method 500.

At step 502, network adapter 110A determines an update to an aspect of the operation of itself and/or an aspect of the operation of medical data source 102A. For example, network adapter 110A may determine an increment to the uptime of the network adapter 110A or the medical data source 102A. The network adapter 110A may count an amount of time since the network adapter 110A or medical data source 102A was powered on and may determine an increment of uptime at predefined intervals (e.g., every hour). The network adapter 110A may determine an increment to the number of uses of the network adapter 110A or the medical data source 102A. For example, the network adapter 110A may detect a start of medical data transmission from the medical data source 102A and may determine that the number of uses should be incremented or the network adapter 110A may detect a power-on of the medical data source 102A or the network adapter 110A and may determine that the number of uses should be incremented.

The update to an aspect of the operation of the network adapter 110A that may be determined at step 502 may include a data communication signal power of the network adapter 110A and/or a signal-to-noise ratio of the connection of the network adapter 110A to the network 106. For example, the network adapter 110A may be configured to determine the power of the signal generated by its transceiver (e.g., transceiver 316 of FIG. 3) and/or the signal-to-noise ratio of the connection between its transceiver and the network 106.

At step 504, the update to the aspect of the operation of the network adapter 110A and/or medical data source 102A may be stored in a memory. The memory may be a memory of the network adapter 110A, in which case the network adapter 110A may perform step 504 by updating its memory to reflect the update. Alternatively, the update to the aspect of the operation of the network adapter 110A may be stored in a memory of the medical room control system 108 and/or a memory of remote computing system 114. In such a case, step 504 may include the network adapter 110A sending the update to the medical room control system 108, and the medical room control system storing the update in its memory or transmitting the update to the remote computing system 114 for remote storage. The update may be stored as an update to one or more attributes of the network adapter 110A or medical data source 102A stored in an attribute database.

At step 506, a notification may be provided to a user based on the update to the aspect of the operation of the network adapter 110A or the medical data source 102A. The notification may indicate that a maintenance associated with the network adapter 110A and/or medical data source 102A should be conducted. For example, a number of uses and/or device uptime may exceed a predetermined threshold associated with a planned maintenance, and a notification may be provided indicating that the maintenance should be performed. Step 506 may be performed, for example, by medical room control system 108 and/or remote computing system 114 based on an update received from network adapter 110A. For example, the number of uses of network adapter 110A may be sent to medical room control system 108, medical room control system 108 may compare the number of uses to a predetermined threshold, and if the threshold is exceeded, may provide a notification (e.g., a visual notification on a display of the medical room control system 108) indicating that the network adapter 110A and/or medical data source 102A should be serviced. Alternatively, the medical room control system 108 may provide the update to a remote computing system 114, which may do the comparison with a threshold and provide the notification (e.g., an email notification to maintenance personnel) accordingly.

As another example, the update to the aspect of the operation of the network adapter 110A may correspond to a signal-to-noise ratio of the connection between the network adapter 110A and the network 106, and step 506 may include the medical room control system 108 or the remote computing system 114 providing a notification (e.g., a notification displayed on a display and/or an email or text notification to maintenance personnel) associated with the signal-to-noise ratio dropping below a predetermined threshold (i.e., too much noise). The drop in signal-to-noise ratio may be associated with a deterioration of one or more components of network 106 (such as a deterioration of a fiber optic cable connecting the network adapter 110A to network 106), and the notification provided at step 506 may indicate that maintenance to the network component(s) is needed (e.g., a fiber optic cable should be replaced).

Thus, method 500 may enable automatic monitoring of one or more aspects of the operation of medical data sources, medical data sinks, and/or network adapters, which may increase uptime of a given medical room and/or reduce the amount of time and/or effort that maintenance personnel need to expend to ensure medical room uptime. A medical room control system could use method 500 to monitor which devices are connected within the medical room and their uptimes and/or number of uses. Such information could be aggregated for multiple medical rooms and other areas of a medical facility and/or medical facility network (e.g., a medical room control system could report its monitoring to a central server) to provide complete visibility into the medical data source and sink install base.

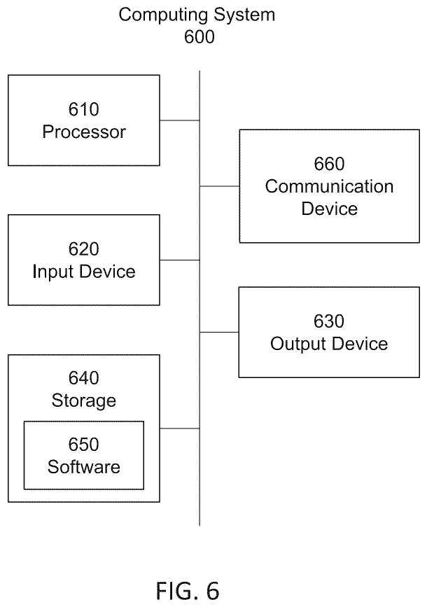

FIG. 6 illustrates an example of a computing system 600 that may be used in any one of the systems described herein, such as medical data source 102, medical data sink 104, network adapter 110, medical room control system 108, and/or remote computing system 114 of FIG. 1. System 600 can be a computer connected to a network. System 600 can be a client computer or a server. As shown in FIG. 6, system 600 can be any suitable type of microprocessor-based system, such as a personal computer, workstation, server, or handheld computing device (portable electronic device) such as a phone or tablet. The system can include, for example, one or more of a processor 610, input device 620, output device 630, storage 640, and communication device 660. Input device 620 and output device 630 can generally correspond to those described above and can either be connectable or integrated with the computer.

Input device 620 can be any suitable device that provides input, such as a touch screen, keyboard or keypad, mouse, gesture recognition component of a virtual/augmented reality system, or voice-recognition device. Output device 630 can be or include any suitable device that provides output, such as a touch screen, haptics device, virtual/augmented reality display, or speaker.

Storage 640 can be any suitable device that provides storage, such as an electrical, magnetic, or optical memory, including a RAM, cache, hard drive, removable storage disk, or other non-transitory computer readable medium. Communication device 660 can include any suitable device capable of transmitting and receiving signals over a network, such as a network interface chip or device. The components of the computer can be connected in any suitable manner, such as via a physical bus or wirelessly.

Software 650, which can be stored in storage 640 and executed by processor 610, can include, for example, the programming that embodies the functionality of the present disclosure (e.g., as embodied in the devices as described above). For example, software 650 can include one or more programs for performing one or more of the steps of method 200, method 400, and/or method 500.

Software 650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

System 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 600 can implement any operating system suitable for operating on the network. Software 650 can be written in any suitable programming language, such as C, C++, Java, or Python. In various aspects, application software embodying the functionality of the present disclosure can be deployed in different configurations, such as in a client/server arrangement or through a Web browser as a Web-based application or Web service, for example.

The foregoing description, for the purpose of explanation, has been described with reference to specific aspects. However, the illustrative discussions above are not intended to be exhaustive or to limit the invention to the precise forms disclosed. Many modifications and variations are possible in view of the above teachings. The aspects were chosen and described in order to best explain the principles of the techniques and their practical applications. Others skilled in the art are thereby enabled to best utilize the techniques and various aspects with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for controlling use of medical data comprising, at a medical room control system:
querying a network adapter via a communication network to obtain information stored at the network adapter, the network adapter configured to encode medical data generated by a medical data source for transmission via the communication network and/or decode medical data received via the communication network for use by a medical data sink;
determining at least one attribute associated with the network adapter, the medical data source, or the medical data sink based on the information stored at the network adapter; and
controlling use of the medical data by the medical data sink based on the at least one attribute.

2. The method of claim 1, wherein controlling the use of the medical data by the medical data sink comprises configuring the medical data source and/or the medical data sink based on the at least one attribute.

3. The method of claim 2, wherein configuring the medical data source and/or the medical data sink comprises configuring a resolution and/or a color space of the medical data sink.

4. The method of claim 1, wherein determining the at least one attribute comprises extracting an identifier for the network adapter, the medical data source, or the medical data sink from the information stored at the network adapter.

5. The method of claim 4, wherein the at least one attribute comprises an association of the medical data source with the medical data sink, and wherein controlling the use of the medical data by the medical data sink based on the at least one attribute comprises updating a routing table based on the association of the medical data source with the medical data sink so that the medical data from the medical data source is routed to the medical data sink.

6. The method of claim 4, wherein the network adapter is associated with the medical data source, and the at least one attribute comprises an association of the network adapter associated with the medical data source with a network adapter associated with the medical data sink, and wherein controlling use of the medical data by the medical data sink based on the at least one attribute comprises updating a routing table based on the association of the network adapter associated with the medical data source with the network adapter associated with the medical data sink so that the medical data from the medical data source is routed to the medical data sink.

7. The method of claim 1, wherein determining the at least one attribute comprises extracting the at least one attribute from the information stored at the network adapter.

8. A system for controlling display of medical video comprising one or more processors and memory storing one or more programs for execution by the one or more processors, the one or more programs including instructions that when executed by the one or more processors cause the system to:
query a network adapter via a communication network to obtain information stored at the network adapter, the network adapter configured to encode medical data generated by a medical data source for transmission via the communication network and/or decode medical data received via the communication network for use by a medical data sink;
determine at least one attribute associated with the network adapter, the medical data source, or the medical data sink based on the information stored at the network adapter; and
control use of the medical data by the medical data sink based on the at least one attribute.

9. The system of claim 8, wherein the system comprises the network adapter, the medical data source, and the medical data sink.

10. The system of claim 8, wherein controlling the use of the medical data by the medical data sink comprises configuring the medical data source and/or the medical data sink based on the at least one attribute.

11. The system of claim 10, wherein the medical data source and/or the medical data sink is configured based on a user profile.

12. The system of claim 10, wherein configuring the medical data source and/or the medical data sink comprises configuring a resolution and/or a color space of the medical data sink.

13. The system of claim 8, wherein determining the at least one attribute comprises extracting an identifier for the network adapter, the medical data source, or the medical data sink from the information stored at the network adapter.

14. The system of claim 13, wherein determining the at least one attribute comprises using the identifier to query a database to obtain the at least one attribute, wherein the database is hosted by a remote computing system or is stored locally at the medical room control system.

15. The system of claim 13, wherein the at least one attribute comprises an association of the medical data source with the medical data sink, and wherein controlling the use of the medical data by the medical data sink based on the at least one attribute comprises updating a routing table based on the association of the medical data source with the medical data sink so that the medical data from the medical data source is routed to the medical data sink.

16. The system of claim 13, wherein the network adapter is associated with the medical data source, and the at least one attribute comprises an association of the network adapter associated with the medical data source with a network adapter associated with the medical data sink, and wherein controlling use of the medical data by the medical data sink based on the at least one attribute comprises updating a routing table based on the association of the network adapter associated with the medical data source with the network adapter associated with the medical data sink so that the medical data from the medical data source is routed to the medical data sink.

17. The system of claim 13, wherein the identifier comprises a Global Trade Item Number (GTIN).

18. The system of claim 8, wherein determining the at least one attribute comprises extracting the at least one attribute from the information stored at the network adapter.

19. The system of claim 8, wherein the one or more programs include instructions for tracking a number of uses and/or a device uptime associated with the network adapter and generating a notification indicating maintenance associated with the network adapter based on the number of uses and/or the device uptime.

20. A non-transitory computer readable medium storing instructions for execution by one or more processors of a computing system to cause the computing system to:
query a video network adapter via a communication network to obtain data stored at the video network adapter, the video network adapter configured to encode medical video data generated by a medical video source for transmission via the communication network or decode medical video data received via the communication network for display by a medical video sink;
determine at least one attribute associated with the video network adapter, the medical video source, or the medical video sink based on the data stored at the video network adapter; and
control display of the medical video data by the medical video sink based on the at least one attribute.
